(19) United States
(12) Patent Application Publication
(10) Pub. No.: US 2026/0038687 A1
(43) Pub. Date: Feb. 5, 2026

(54) HEALTH CARE PREDICTION AND WARNING SYSTEM AND OPERATING METHOD THEREOF

(71) Applicant: HAO-YI FAN, Taipei City (TW)

(72) Inventor: HAO-YI FAN, Taipei City (TW)

(21) Appl. No.: 19/287,844

(22) Filed: Aug. 1, 2025

(30) Foreign Application Priority Data
Aug. 2, 2024 (TW) 113128872

(51) Int. Cl. G16H 50/20; G16H 40/67; G16H 50/30; G16H 80/00

(52) U.S. Cl. CPC G16H 50/20; G16H 40/67; G16H 50/30; G16H 80/00

(57) ABSTRACT

A health care prediction and warning system and an operating method thereof are provided. The system is implemented by a cloud system which operates a care-taking warning prediction model. The cloud system receives physiological data from various terminal devices. After analyzing the physiological data, analyzed result is compared with criteria set by the cloud system for determining whether the physiological data is sufficient to predict physiological status of a care recipient. Under first criterion, a result of predicted physiological status of the care recipient is generated based on the received physiological data; under second criterion, the cloud system requires the terminal device to transmit more data in different time period for effectively predicting the physiological status; and, under third criterion, the cloud system requires the terminal device to provide other sensing data generated by other sensors so as to effectively predict the physiological status.

### CROSS-REFERENCE TO RELATED PATENT APPLICATION

This application claims the benefit of priority to Taiwan Patent Application No. 113128872, filed on Aug. 2, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a method for managing physiological status of a care recipient via a cloud system, and more particularly to provide a health care prediction and warning system that is able to determine physiological status based on physiological data received from a terminal and a method for operating the system.

### BACKGROUND OF THE DISCLOSURE

The conventional medical care system operates mainly by setting up physiological sensors on or around a care recipient, and the physiological sensors are connected to a care center through a network which allows the care center to obtain the physiological information transmitted by the terminal physiological sensor at all times, such that a physiological status of the care recipient can be determined.

Moreover, whether or not the conventional medical care system can achieve the purpose of caring for the care recipient depends on the immediate determination ability of the care center and the physiological data provided by the physiological sensor of the care recipient. However, since the physiological sensors are often worn on the care recipient, the physiological sensors can only provide limited physiological information, and various physiological data need to be independently interpreted. In addition, the method for accurately determining the physiological status should also be based on past medical records of the care recipient, current environmental factors, and the collected physiological data, such that the physiological status can be comprehensively determined. The current technology is incapable of effectively implementing the method for accurately determining the physiological status.

Furthermore, regarding various types of the physiological data, physicians are required to make decisions based on their experiences, which cannot account for all of the possibilities. Therefore, effectively generating warnings or reminders for specific situations are not possible, which makes home care inconvenient. Moreover, especially for home care purposes, the physiological sensors are generally incomparable with the equipment at medical institutions, and the physiological data obtained therefrom is limited and cannot be used for an accurate interpretation of specific conditions, such as the sleep quality of the care recipient. The sleep quality of the care recipient is an important issue that affects home care. Sleep quality not only affects the health condition of the care recipient, but also affects the quality of life of the caretaker, since the caretaker has to pay attention to the condition of the care recipients at all times.

Therefore, an artificial intelligence (AI) technology can be of help for the purpose of home care, since it can assist in developing an effective health management method under a circumstance where there is limited terminal equipment for the needs of the caregiver for health management, and avoiding unnecessary waste of medical resources. At present, conventional technologies still lack a health management system that can be applied to most of the care recipients.

### SUMMARY OF THE DISCLOSURE

The disclosure relates to a health care prediction and warning system and a method for operating the system. The method is operated in a central server. A connection between the central server and a terminal device is established. The central server continuously or periodically receives the physiological data transmitted by the terminal device via the connection. Preferably, the central server continuously receives the continuous physiological data that is received in multiple stages or by time sharing. The terminal device itself can be a sensor device or connects with one or more sensors that continuously provide physiological data and environmental data. In the central server, after the physiological data is analyzed, features of the physiological data can be obtained and used for confirming physiological status of the care recipient. The features of the physiological data can also be referred to for determining whether the physiological status of the care recipient meets any of the criteria set in the central server.

When the central server determines that additional data is needed based on the features of the physiological data, an instruction for requesting the additional data is generated and transmitted to the terminal device for driving the terminal device to transmit the additional data to the central server. The additional data allows the central server to effectively determine physiological status of the care recipient by a care-taking warning prediction model operated in the central server or through prediction indicators that are learned by the central server. In the central server, the care-taking warning prediction model is operated to acquire features of the continuously-received physiological data at every time point or at every time period, and calculate probabilities of various health risks so as to determine physiological status of the care recipient.

Further, the central server relies on a preset decision rule to decide a flow to be operated in compliance with one of multiple criteria meeting the physiological status of the care recipient according to features of the physiological data. The criteria at least include a first criterion for generating a result of predicting physiological status of the care recipient through multiple prediction indicators or by the care-taking warning prediction model based on the physiological data; a second criterion for the central server to request the terminal device to transmit the physiological data at another time period according to the result that is generated through multiple prediction indicators or by the care-taking warning prediction model; and a third criterion for the central server, based on the result that is generated through the multiple prediction indicators or by the care-taking warning prediction model, to request the terminal device to provide a sensing data generated by another sensor, so as to effectively predict the physiological data of the care recipient.

Further, for the additional data that the terminal device transmits to the central server, the central server synchronously labels the continuous data and non-continuous data before and after the time to request for the additional data, or synchronously labels the continuous data and the non-continuous data that are obtained at the same time period. Thus, the data labeled by the central server is an individualized data for the care recipient, and an individualized care-taking warning prediction model can be established by learning the features of the individualized data through an artificial intelligence technology.

Further, the terminal device can implement one of the edge-computing devices of the health care prediction and warning system, and the multiple prediction indicators or the care-taking warning prediction model that is individualized can be obtained through a learning process by the central server. The physiological status of the care recipient can be initially determined by the terminal device.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

FIG. 1 is a schematic diagram depicting a circumstance for operating a health care prediction and warning system according to one embodiment of the present disclosure;

FIG. 2 is a schematic diagram illustrating a system framework of the health care prediction and warning system according to one embodiment of the present disclosure;

FIG. 3 is a schematic diagram depicting an AI module operated in the health care prediction and warning system according to one embodiment of the present disclosure;

FIG. 4 is a flowchart illustrating application operations the health care prediction and warning system under criterion according to one embodiment of the present disclosure;

FIG. 5 is another flowchart illustrating data collection and application of the health care prediction and warning system according to another embodiment of the present disclosure;

FIG. 6 is a flowchart illustrating an operating method of the health care prediction and warning system among sensors, terminal devices and a central server according to one embodiment of the present disclosure; and

FIG. 7 is one further flowchart illustrating the operating method of the health care prediction and warning system among sensors, terminal devices and the central server according to one further embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

Since current care systems mostly rely on human resources (e.g., the caregivers) to conduct care on the care recipients and may need to provide 24-hour whole day care services in certain circumstances, it puts a huge burden on the home caregivers and also causes high pressure on manpower requirements in medical institutions. Even though the modern care system incorporates various sensors to assist in the care services, the care system still requires the manpower to frequently pay attention to the care recipient for preventing the care system from failing to accurately warn of a real emergent event or causing the caregivers to frequently resolve erroneous messages caused by the false alarms due to it lacks a care warning mechanism specified to the personal physiological and environmental circumstance of the care recipient. Furthermore, the conventional physiological sensor disposed at the care recipient's location is usually a sensor that provides a single or limited amount of information and is not sufficient to effectively predict the physiological status of the care recipient, so that the current care system fails to effectively predict the physiological status of the care recipient. Thus, provided in the present disclosure is a health care prediction and warning system that provides the sensors with multimodal sensing capabilities and services of prediction of physiological events, and a method for operating the system. The health care prediction and warning system incorporates a machine-learning technology to establish a care-taking warning prediction model that provides automatic and individualized terminal care and warning services via a cloud server. One of the main technical objectives of the health care prediction and warning system of the present disclosure is to allow a terminal device that is only equipped with a sensor having a single function or a few sensors to obtain a relatively comprehensive health care. The health care prediction and warning system can relieve the burden of the caregiver and also solve the problems of traditional manpower or sensors with limited sensing functionality.

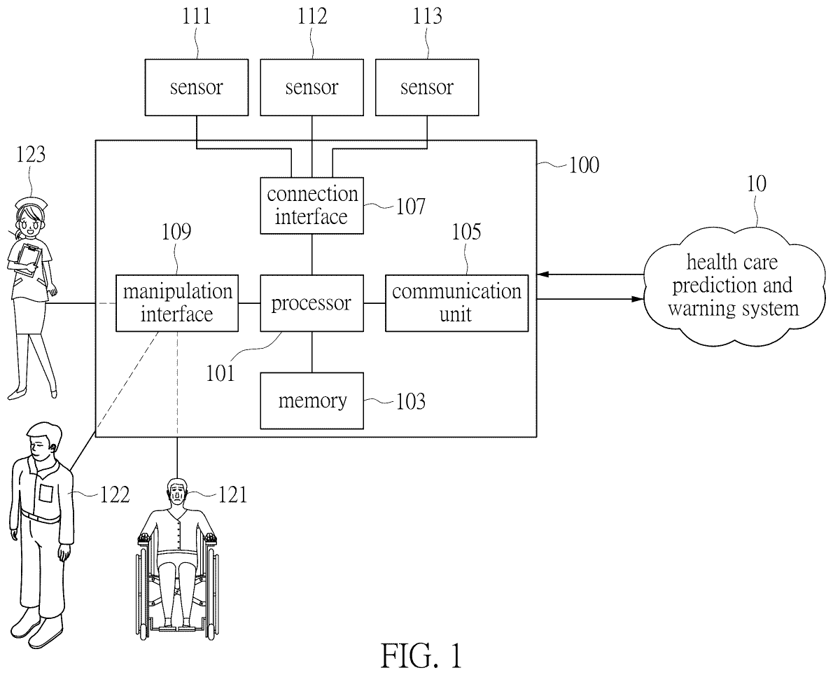

FIG. 1 is a schematic diagram depicting a circumstance operating the health care prediction and warning system according to one embodiment of the present disclosure.

The diagram shows a health care prediction and warning system 10 that can be implemented through collaboration of software and hardware of a computer system. The health care prediction and warning system 10 embodies a cloud care service that allows a terminal device that is only with limited physiological sensing capabilities to obtain relatively complete health care functionalities. In certain embodiments of the present disclosure, in the health care prediction and warning system 10, an artificial intelligence technology is used to learn correlations between features of various physiological data and diseases for establishing a care-taking warning prediction model. In addition to continuous data (e.g., physiological data, environmental data or others) that are continuously or periodically (e.g., in multiple stages or by time sharing) provided by the terminal device, the health care prediction and warning system 10 also obtains non-continuous data (e.g., physiological data, environmental data or others) that is meaningful but limited information received from the terminal device in accordance with setting or specified criteria, so that the care-taking warning prediction model is able to effectively predict a physiological trend of the care recipient and provide a warning message.

It should be noted that the health care prediction and warning system 10 establishes the care-taking warning prediction model by learning the continuous data or the non-continuous data as a generalized prediction model suitable for all persons. Further, the care-taking warning prediction model can also be categorized to be an individualized model; for example, the care-taking warning prediction model can be categorized into different categories suitable for different races, different regions, different diseases or the like.

A terminal device 100 shown in the diagram is such as a user-end computer system such as a smart phone, a tablet computer or a desktop computer. Hardware components of the terminal device 100 include a processor 101, a memory 103, a display or a display panel (not shown in the diagram), a communication unit 105 connected with an external system, a connection interface 107 used to connect with various sensors (111, 112, 113) via a wired or wireless connection, and a manipulation interface 109 that is provided for a user (e.g., a caregiver 123, a care recipient 121 or a designated related person 122) to setup and issue a message. It should be noted that the terminal device 100 can be an intermediate device for transmitting data between sensors and the health care prediction and warning system 10 and can also be a sensor that is used for sensing specific information. For example, the terminal device 100 can be an accelerometer, a photographing module or a specific electronic device that is used to sense environmental information such as slights and temperatures.

According to one embodiment of the present disclosure, the caregiver 123 is such as any of family members, relatives, friends, medical staff or employed caregivers who care for the care recipient 121, the care recipient 121 can be a patient under care staying at home or in a care center, and a designated related person 122 can be a specific person who is set for the health care prediction and warning system 10 or in the terminal device 100. The manipulation interface 109 provided by the terminal device 100 can be a physical emergent button, a user interface provided by the electronic device, or a software interface initiated by a handheld device for the caregiver 123, the care recipient 121 or the designated related person 122 to generate a message transmitted to the health care prediction and warning system 10 under a specific circumstance. For example, when the caregiver 123 finds an abnormal condition occurred to the care recipient 121, the caregiver 123 can issue an emergent message via the manipulation interface 109, or the care recipient 121 can also issue the message when he feels some unusual condition with his body via the manipulation interface 109.

The communication unit 105 of the terminal device 100 is used to perform a wired or wireless communication protocol for establishing a connection between the terminal device 100 and the health care prediction and warning system 10. The connection interface 107 of the terminal device 100 is a wired interface (e.g., a universal serial bus (USB) or any of industrial standard interfaces) or a wireless interface for a wireless local area network or a Bluetooth communication module that is configured to connect with various peripheral devices or the sensors 111, 112 and 113. Further, the manipulation interface 109 of the terminal device 100 can be an emergent button, a touch-sensitive interface or a computer display interface provided for the caregiver 123, the care recipient 121 and the designated related person 122 to setup functions of the terminal device 100 or to send a warning signal when a specific event occurs. For example, when the caregiver 123 or the designated related person 122 can push the manipulation interface 109 of the terminal device 100 for generating a warning signal when he finds that the care recipient 121 is in an abnormal state, and the warning signal is then transmitted to the health care prediction and warning system 10. In another example, the care recipient 121 can push the manipulation interface 109 to generate a warning signal to be transmitted to the health care prediction and warning system 10 when he feels himself in an abnormal condition.

In certain embodiments of the present disclosure, the terminal device 100 can itself implement a sensor device or electrically connect with one or more sensors (e.g., the sensors 111, 112 and 113 shown in the diagram) so as to implement a terminal care system that is operated on the user's end, e.g., the place of the caregiver 123, the care recipient 121 or the designated related person 122. The sensor device or any of the one or more sensors can be a sensor with a single sensing capability or a sensor with multimodal sensing capabilities. The sensor device or the sensor can be a physiological sensor or an environmental sensor that can be used to sense a heart rate, a blood pressure, a blood sugar, a body temperature, an air temperature, an air index, a breathing status, an activity status, a sound or an image of the care recipient 121 in a contact or a non-contact method. Based on a principle of personal information confidentiality and authorization, the physiological data received by the terminal device 100 should be processed with encoding, encryption and format conversion by a processing circuit of the terminal device 100 and then transmitted to the health care prediction and warning system 10. The processed physiological data transmitted to the health care prediction and warning system 10 includes continuous data that is generated by a continuous sensing process and/or non-continuous data that is additional data being provided by the terminal device 100 in response to a request of the system or the additional data being generated by triggering any of the sensors.

Thus, the sensor device or the one or more sensors can continuously or periodically generate the continuous data or generate the non-continuous data based on criteria set by the system. The continuous data and the non-continuous data are transmitted to the health care prediction and warning system 10 via a connection between the terminal device 100 and the health care prediction and warning system 10. The terminal device 100 receives signals generated by the health care prediction and warning system 10 via the communication unit 105. The signals generated by the health care prediction and warning system 10 are such as the warning signals being generated when a specific physiological event is predicted by the care-taking warning prediction model based on a setting made by the system or criteria set by the system, or the signals can be used to request additional physiological data according to an analytical result. Accordingly, the care-taking warning prediction model operated in the health care prediction and warning system 10 can simultaneously rely on the continuous and non-continuous data to predict the physiological status of the care recipient.

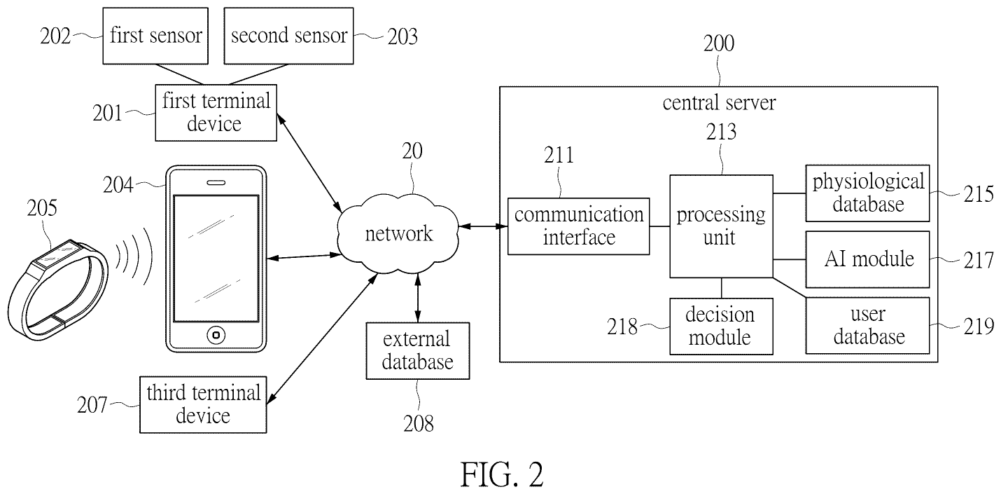

The health care prediction and warning system is preferably a cloud system that can be implemented by a central server. The central server can therefore be a cloud server, still a local server, or a serving program executed in a local device. Reference is made to FIG. 2, which is a schematic diagram depicting a system framework of the health care prediction and warning system according to one embodiment of the present disclosure.

The diagram shows a central server 200 of the health care prediction and warning system, and the main components of the central server 200 include a processing unit 213 that is used to operate intelligent models and software processes of the central server 200 and other circuit and function elements electrically connected with the processing unit 213. The circuit elements are such as a communication interface 211 connected with a network 20, a decision module 218 that is used to decide following steps by a software means according to a result as processing the physiological data, a physiological database 215 storing the physiological data associated with different users, and a user database 219 storing user data. In particular, the central server 200 includes an AI module 217 having a machine-learning algorithm that is performed by the processing unit 213 for learning features of the physiological data. The features of the physiological data are referred to for training a care-taking warning prediction model that is able to predict the physiological status of the care recipient based on the features of the physiological data. In one of the embodiments of the present disclosure, the decision module 218 of the central server 200 relies on a preset decision rule to decide following steps according to an analytic result of the physiological data. For example, in the central server 200, when the physiological features extracted from the physiological data are found to be within a normal range, the received data can be discarded; when additional data are determined to be required based on the analytic result of the physiological data, the processing unit 213 of the central server 200 generates a corresponding request instruction for requesting the additional data from the terminal device so as to determine physiological status of the care recipient; when sensing data generated by a specific sensor is determined to be required based on the physiological data, the processing unit 213 also generates a corresponding request instruction. The request instruction is used to request the terminal device to provide the sensing data generated by the sensors, a specific sensor or the sensing data generated at a specified time period. The central server 200 is able to integrate multiple types of data to determine the physiological status of the care recipient. The additional data can be regarded as a sensing data that the decision model 218 of the central server 200 determines which it requires other than the instant physiological data in a process of processing the physiological data. For example, the additional data can be the sensing data to be generated by the same sensor at another time period or the sensing data to be generated by different sensors at the same time period or another specified time period.

It should be noted that the end devices such as a first terminal device 201, a first sensor 202, a second sensor 203, a second terminal device 204, and a third sensor 205, and a third terminal device 207 embody the IoTs (Internet of Things) of the health care prediction and warning system. These IoT devices can be communicated with each other for integrating sensing data so as to achieve a practical application such as implementing a terminal care system through collaboration of artificial intelligence technologies.

The terminal device connected with the central server 200 via the network 20 can be a computer device with communication capability and can be used to connect with various terminal sensors. As shown in the diagram, the first terminal device 201 that can be an edge-computing device connects with the first sensor 202 and the second sensor 203, and both the first sensor 202 and the second sensor 203 can generate the physiological data of the care recipient and sensing data of the care recipient's environment. The terminal device connected with the central server 200 can be the second terminal device 204 that connects with the third sensor 205 worn on the care recipient for acquiring the physiological data generated by the third sensor 205 at any time, and the physiological data is transmitted to the central server 200 via the network 20.

In the embodiment, the terminal device connected with the central server 200 can be the third terminal device 207 that can also be an edge-computing device with communication capability. In another aspect, the third terminal device 207 is such as an environmental sensor that is disposed at a specific place for sensing physical or chemical data. For example, the environmental sensor can be used to sense temperature, humidity, air pressure, images, sounds, air quality (e.g., PM2.5/10) and/or air composition (e.g., carbon monoxide and/or carbon dioxide), etc. These environmental data that can be continuous data or non-continuous data meeting a specific criterion can also be transmitted to central server 200. The correlated data sections among the data can be labeled and saved. The sensing data within another time period requested by the central server 200 can be provided for the care-taking warning prediction model operated in the central server to perform prediction and judgment based on the more complete data.

Further, the central server 200 can connect with an external database 208 via the network 20. The external database 208 generally indicates any source from which available data can be obtained. For example, the external database 208 can be an internal medial institute having a lot of references with medical evidence that can be referred to for updating blood pressure standards, blood oxygen and heart rate standards. The external database 208 may include various physiological data and environmental data covering people of all races, genders, regions and countries that can be referred to be a training dataset and verification database for machine learning. The training database and the verification database allow the central server 200 to train the care-taking warning prediction model with various data and provide an individualized health management solution for different people of races, genders, regions and countries.

Thus, the central server 200 can connect with terminal care systems that are implemented by the various types of terminal devices. The terminal care system can include a physiological sensor, an audio receiver, an image sensor and an environmental sensor and can be used to perform analysis on the physiological and environmental data relating to the care recipients at different ends and make decision. Finally, the care-taking warning prediction model can be used to predict the physiological status of the care recipient so as to achieve the purpose of intelligent care and warning.

In an exemplary example of the terminal care system, the terminal care system is disposed at the care recipient's end and its main components are such as various sensors around the care recipient. The sensors are such as various types of physiological sensors, audio receivers, image sensors and environmental sensors. The terminal care system also provides the caregiver or the care recipient an operable device that allows the central server 200 to directly receive messages inputted by the care recipient or the caregiver. For example, the terminal care system receives the messages inputted by the care recipient or the caregiver through various terminal devices and the messages become feedback messages that are fed back to the health care prediction and warning system while training the care-taking warning prediction model. Further, the central server 200 that implements the health care prediction and warning system can connect with an external system or an external medical cloud via a specific communication protocol so as to acquire various types of clinical data or big data analytical results of individuals or groups. These data can be used as training dataset for health management by an artificial intelligence technology.

It should be noted that the health care prediction and warning system can adopt a hybrid framework that includes the above-mentioned terminal devices that can be the edge-computing devices and the central server 200. The terminal devices with edge-computing power can perform part of operations that are originally configured to be run by the central server 200, for example, some pre-processing processes can be performed by the edge-computing devices for mitigating loading of the central server 200, reducing network latency and improving privacy.

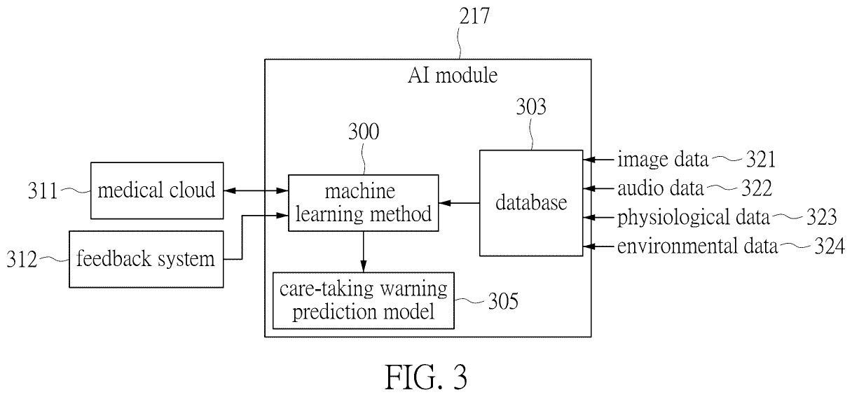

The care-taking warning prediction model operated in the central server 200 can be obtained by a machine-learning algorithm performed by the AI module 217. Reference is made to FIG. 3, which is a schematic diagram illustrating the AI module of the health care prediction and warning system according to one embodiment of the present disclosure.

The AI module 217 which can also be operated in an edge-computing device can be implemented through collaboration of software and computing power of hardware in the health care prediction and warning system. The processing unit 213 of the central server performs a machine learning method 300 that relies on data (i.e., the physiological data 323 and environmental data 324) provided by the health care prediction and warning system to train the care-taking warning prediction model for identifying correlations of the data and achieving purpose of prediction of physiological status.

In certain embodiments of the present disclosure, a database 303 of the health care prediction and warning system is used to store the various types of data such as the image data 321 retrieved from the terminal device that takes pictures of the care recipient, the audio data 322 that is obtained by using an audio receiver to record sounds of the care recipient or the environment, the physiological data 323 that is generated by the various types of physiological sensors that are used to sense physiological status of the care recipient, and the environmental data 324 sensed by the various types of environmental sensors. The machine learning method 300 is performed to acquire a variety of sensing data from the database 303 for preventing training the care-taking warning prediction model 305 with poor accuracy from only learning single or overly simplistic sensing data. It should be noted that the accuracy of AI-based physiological status prediction for the care recipient can be improved by acquiring various types of the physiological and environmental data, and furthermore a decision-making mechanism of the health care prediction and warning system is beneficial to predict the physiological status of the care recipient more accurately while requesting additional data from the terminal device based on the limited sensing data.

When the health care prediction and warning system is in operation, the continuous data or the non-continuous data received from the terminal device can be retrieved from the database 303 of the health care prediction and warning system, and the machine learning method 300 is used to learn the features of the continuous data or the non-continuous data so as to train the care-taking warning prediction model 305 that is configured to predict the physiological status of the care recipient.

The image data 321 in the database 303 includes the images retrieved from the various sensors. The images include the posture (e.g., the image data 321) of the care recipient, for example, the posture such as turning over, the movement of hands and feet. Furthermore, the physiological data, such as chest fluctuation, frequency, depth, etc., can be obtained. A recognition ability of the posture images can be established after training, and correlations between the posture images of the care recipient and other data can also be established. The database 303 can obtain sound data 322, such as daily routine and sleep of the care recipient, and the sound data 322 can be identified as specific physiological reactions, such as a sound of having sputum, a coughing sound, and a sound generated by the phenomenon of respiratory suspension (or respiratory distress), etc., and the correlations between the recognized sound and other data can be established. The database 303 can obtain physiological data 323, such as body temperature, heart rate, respiration, pulse, blood oxygen, etc., of the care recipient, and in addition to the normal and abnormal conditions of the physiological data, establishing the correlations between any time being and other data are required. The database 303 can also obtain the environmental data 324, through detecting the environmental data of the care recipient through the environmental sensors (which can be deemed as the first group of sensors or the second group of sensors) the environmental data becomes one of the factors of the care-taking warning prediction model 305 established by the machine learning method 300. The data can include information, such as temperature and humidity, air quality, climate, daily routine, etc., related to the care recipient, and can also establish correlation with other data through learning. The data are then analyzed, so as to find an overall correlation among the data.

In particular, an individualized care-taking warning prediction model 305 can be established when the artificial intelligence applied to the health care prediction and warning system adopts the above-mentioned individualized image data 321, the audio data 322, the physiological data 323 and the environmental data 324.

After that, the machine learning method 300 being used cooperatively with the historical and real-time data collected by the database 303, and being used cooperatively with the actual recorded physiological response is able to establish personalized rules, so as to establish the care-taking warning prediction model 305 for predicting the physiological condition of the care recipient. With operation of the care-taking warning prediction model 305, what may possibly happen can be predicted based on the data before any dangerous event should occur, and a purpose of preventing and warning in advance can be achieved.

According to one embodiment of the present disclosure, in addition to obtaining the sensing data generated by the terminal device, the machine learning method 300 performed in the AI module 217 can obtain more comprehensive physiological data from a medical cloud 311 having the sensing data relating to many people, by which the physiological data can be used to train the care-taking warning prediction model 305. For example, the AI module 217 can obtain de-identified clinical data of various groups from the medical cloud 311, and in addition to providing data required for training, the de-identified clinical data can also be used to verify the care-taking warning prediction model 305.

The system further includes a feedback system 312. The AI module 217 receives the feedback information from the care recipient, the caretaker, or generated through other methods through a feedback system 312. As mentioned in the above embodiments, the health care prediction and warning system has the user interface that can be used to receive the warning notification of the care recipient or other people. The warning notification generated by the user device becomes one of the factors for the machine learning method 300 to establish the care-taking warning prediction model 305. The data are used to verify the prediction model and are used to adjust the parameters of the care-taking warning prediction model 305 in the AI module 217, so that the care-taking warning prediction model 305 can be optimized.

In this way, the machine learning method 300 arranged in the AI module 217 integrates various types of the data obtained by the health care prediction and warning system through the data processing unit, and the machine learning method 300 can set different weights for different types of the data according to the requirements thereof, as well as the personalized data and the data from the medical cloud 311 and the feedback system 312. The data allows the health care prediction and warning system to perform big data analysis so as to establish the care-taking warning prediction model 305. The are-taking warning prediction model 305 is used to determine whether or not the condition of the care recipient reaches a specific warning threshold, and the warning threshold is also a criterion that is referred to for generating the warning notification.

It is worth mentioning that the medical cloud 311 can also obtain terminal data from the health care prediction and warning system. Thus, the information with de-identified labels can be obtained through computation performed in the medical cloud 311. The information that is used to establish the data with the labels is such as time information, identity information, and the related physiological data, and environmental data can be collected simultaneously. The data with labels are apparently correlated in the medical cloud 311 and the AI module 217 of the health care prediction and warning system.

The physiological data and various types of the data that are labeled are one of the effective data that are required to be learned through the artificial intelligence technology, so that the model (e.g., the care-taking warning prediction model) to be established can identify and predict the physiological information. When the model operates, machine learning and verification are still in progress so as to constitute a more comprehensive medical cloud 311.

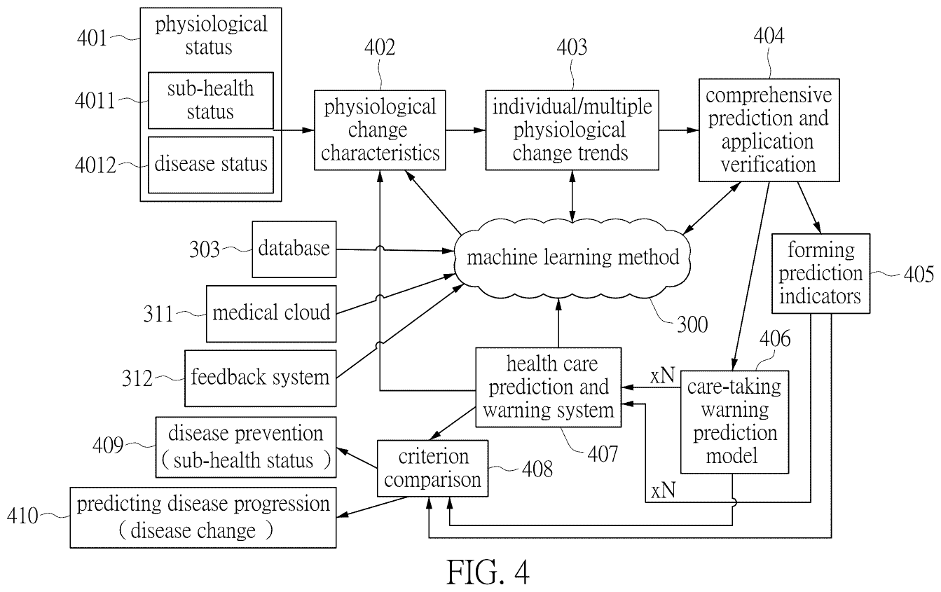

Reference is made to FIG. 4, which is a flowchart illustrating an application process of the health care prediction and warning system with the criteria according to one embodiment of the present disclosure.

The health care prediction and warning system operates the machine learning method 300 through the central server. The machine learning method 300 includes various intelligent learning methods and uses the meaningful data as the training data that covers the data (in the database 303) that is continuously generated by the external medical cloud 311, the sensors at the care recipient's end and the data (from the feedback system 312) that is generated by a triggering action made by the care recipient or the caregiver. Further, the data can also the data that predicted and verified by the care-taking warning prediction model 406 operated in the central server. The care-taking warning prediction model can be optimized by repeatedly trained through the continuous and the non-continuous data provided by the sensors at the care recipient's end, so that the health care prediction and warning system 407 can be implemented.

When the machine-learning algorithm is in operation, the clinical medical data with respect to the various physiological statuses 401 can be firstly obtained. The clinical medical data includes two kinds of medical data such as the data on a sub-health status 4011 with health concerns but no disease and the data on a disease status 4012 that the disease has already occurred. The physiological change characteristics 402 can be obtained by analyzing the above-mentioned data and also form a physiological comprehensive changing trend of a single indicator or multiple indicators over time. The physiological comprehensive changing trend can be used for comprehensive prediction and application verification 404. The machine learning method 300 is also operated for learning the physiological comprehensive changing trend and obtaining the physiological change characteristics 402 for continuously forming an individual or multiple physiological changing trends 403 based on the physiological data transmitted by the terminal devices. The machine learning method 300 operated in the central server the proceeds comprehensive prediction and application verification 404. At last, the central server relies on a result of the prediction and a follow-up verification to form multiple prediction indicator 405 or to train a care-taking warning prediction model 406. For example, the physiological change characteristics 402 with respect to one of physiological indicators such as a heart rate, a blood oxygen, a respiratory rate, a body temperature, a blood pressure and an action can reflect a physiological criterion of a specific disease status and can be labeled. The machine learning method 300 can be repeatedly performed to obtain an evolution or a changing trend 403 of the one or more physiological indicators of a specific disease status. The above-mentioned comprehensive prediction and application verification 404 can be referred to for verifying the various correlations and allocating weights, so that a new scoring system or new prediction indicators 405 can be generated for predicting more disease changes. On the other hand, a result of the comprehensive prediction and application verification 404 can be referred to for establishing or updating the care-taking warning prediction model 406.

Repeating the above process can learn the training dataset that are formed by receiving the data in the various aspects and continuously optimize the care-taking warning prediction model 406 so as to achieve the health care prediction and warning system 407. The prediction indicator 405 or the care-taking warning prediction model 406 can be used to implement a generic or an individualized health care prediction and warning system 407. The health care prediction and warning system 407 cooperates with the central server to perform criterion comparison 408 based on various criteria so as to implement care prediction and warning. The health care prediction and warning system 407 can accordingly confirm the physiological status of the care recipient, for example, disease prevention 409 should be performed while the sub-health status of the care recipient is confirmed, or disease progression 410 can be predicted while a subclinical disease change or trend is confirmed.

Through the process of applying the health care prediction and warning system and the criteria, some targets can be implemented as follows. For example, in addition to the data processing capability of the central server, the system can effectively use the data-retrieving capability of the terminal device via the network 20, such as the sensors 202 and 203 of the terminal device 201 can provide sensing data automatically and continuously to the central server, or the terminal device can be driven by the central server to generate additional data. The sensing data or the additional data generated can also be processed and analyzed by the terminal device. When incorporating the clinical medical data, the system can establish a prediction model by learning the features of the data. After that, even if the terminal device can only provide a single or limited amount of the sensing data, the data can still be used for predicting the physiological status effectively. Further, when a high-precision and multi-modal medical equipment is used at the care recipient's end, the health care prediction and warning system with data accuracy and variety can be implemented for predicting physiological status of the care recipient.

Still further, the health care prediction and warning system achieves a goal of cross-platform integration. For example, the health care prediction and warning system integrates external systems such as the medical cloud 311 through standardized transmission protocol and data format, and the system can provide various health data and electronic medical records. Therefore, the system can effectively use the various database 303 having, but not limited to, the sensing data provided by the terminal device other than the database inside the system.

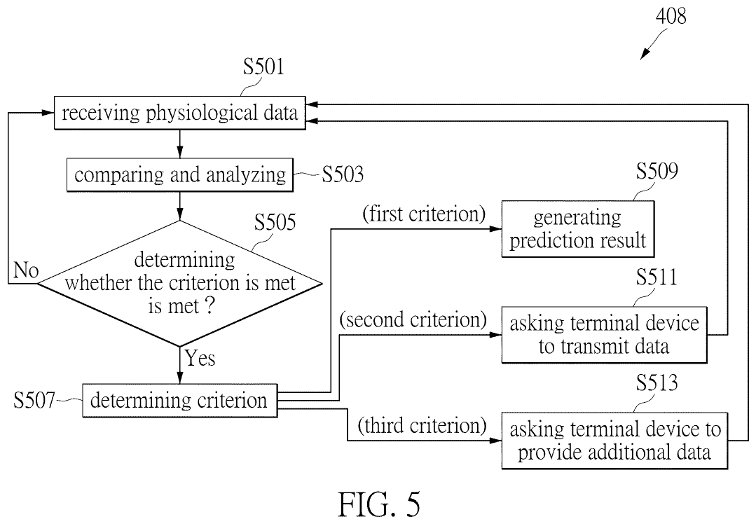

Reference is made to FIG. 5, which is a flowchart illustrating data collection and an application performed in the health care prediction and warning system according to one embodiment of the present disclosure.

A software sequence operated in the health care prediction and warning system (e.g., criterion comparison 408 described in FIG. 4) continuously receives the physiological data of the care recipient from the terminal device or the sensors connected with the terminal device (step S501). Preferably, for implementing a terminal care system, the health care prediction and warning system needs to continuously receive the continuous data that is generated by the terminal device or specified non-continuous data segments. In one of the embodiments, the continuous data generated by the terminal device can be uploaded to the central server continuously, in multiple stages (e.g., uploaded when the data reaches a certain amount), or by time sharing.

In the central server, physiological status of the care recipient is assessed according to physiological data uploaded by the terminal device. In certain embodiments, a care-taking warning prediction model operated in the server is used to obtain features or a changing trend of the physiological data at every time point or in each time interval. The features or the changing trend can be represented by changes in each of scores or indicators and can be used to calculate probabilities of the various health risks. The physiological status of the care recipient can be determined by comparing with the risk thresholds (step S503), and the physiological status can then be confirmed based on the various criteria set in the central servers. Accordingly, one of the multiple criteria set in the central sever can be confirmed (step S505).

If the physiological status of the care recipient does not meet any criterion requesting for additional data (represented by “no”), it indicates that the physiological status being determined by analyzing the physiological data instantly received by the central server is determined to be within a normal range, and therefore the instant physiological data can be ignored. After that, the process goes back to step S501 and the following steps for continuously monitoring and receiving the physiological data from the terminal device. The physiological data includes, but not limited to, the continuous data that is uploaded in multiple stages or by time sharing and the non-continuous data. In addition, the sequence operated in the central server generates instructions to be transmitted to the terminal device based on the criterion met by the physiological status of the care recipient (step S507).

The decision module 218 of the central server 200 shown in FIG. 2 relies on the preset decision rule to decide a following step according to the analysis result of the physiological data. In certain embodiments of the present disclosure, the physiological data processed in the health care prediction and warning system in compliance with a first criterion can be used to determine the physiological status of the care recipient through analysis and comparison based on various prediction indicators or by the care-taking warning prediction model. For example, a prediction result in compliance with the first criterion is generated and outputted if the physiological status may result in diseases or any emergent event (step S509). The prediction result is then transmitted to the terminal device. Afterwards, any follow-up measure can be performed according to the prediction result, for example, calling up an ambulance to pick the care recipient up to a hospital, call a medical staff or notify a designated contact person based on the care recipient's location.

When any result is obtained through the prediction indicators or by the care-taking warning prediction model, the central server (i.e., the central server 200 of FIG. 2) may determine that additional data is still required to confirm the physiological status of the are recipient according to the characteristics of the physiological data. For example, if the current result is insufficient to accurately predict the physiological status of the care recipient, the processing unit of the central server generates a request instruction for additional data according to the decision rule, and the request instruction is transmitted to the terminal device. The terminal device the transmits the additional data (e.g., the physiological data generated in another time period) to the central server for effectively predicting the physiological status of the care recipient. Therefore, the care-taking warning prediction model can proceed prediction in compliance with a second criterion based on a more complete physiological data (step S511). Further, after the prediction is accomplished, the central server can synchronously label the continuous data and the non-continuous data before and after the time to request for the additional data.

If additional data is determined to be required as the central server determines the physiological status of the care recipient through the various prediction indicators or by the care-taking warning prediction model based on the data (i.e., the continuous data and/or the non-continuous data), the processing unit generates the request instruction for requesting for the additional data such as the environmental data relating to the care recipient within the same time period of the physiological data to be generated by other designated sensors within a specific time period, e.g., the same time period or another time period before and after the designated time period from the terminal device. The care-taking warning prediction model operated in the central server can effectively determine the physiological status of the care recipient by integrating the various data in compliance with a third criterion (step S513). Similarly, after the prediction is accomplished, the central server can synchronously label the continuous data and/or the non-continuous that are correspondingly obtained within the same time period or another related time period.

It should be noted that the operations of the health care prediction and warning system are not limited to the above-described criteria. For example, some specific situations may meet both the second criterion and the third criterion, or firstly meet the second criterion for requesting for the data generated by the same sensor at another time period and then meet the third criterion at a next process for requesting for the data generated by another specific sensor at a specified time period. Thus, when the central server obtains the additional data as demands under the second criterion or the third criterion, in accordance with the determination that is made through the prediction indicators or by the care-taking warning prediction model, the first criterion can be finally met so as to effectively predict the physiological status of the care recipient except for requesting for the additional data again.

According to the above-described embodiments of the present disclosure, the additional data being transmitted from the terminal device to the central server can be labeled by a mechanism of the health care prediction and warning system so as to preserve the critical data correlated to the different sensors within a critical time period. Rather than the big data required by the conventional artificial intelligence to establish the models, the health care prediction and warning system of the present disclosure labels the data that are generated by the different sensors within the same or related time periods and makes the data relevant. The correlated data becomes the individualized data for the care recipient. An artificial intelligence technology can learn the correlations among the different data so as to establish an individualized care-taking warning prediction model. The individualized model provides an individualized care service for effectively mitigating the burden on the caregivers.

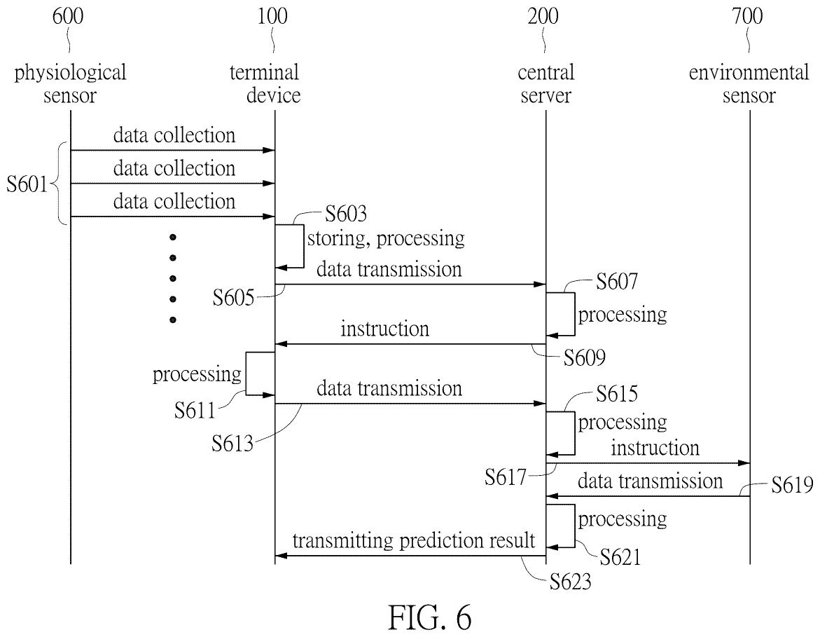

Reference is made to FIG. 5 and in view of FIG. 6, which is a flowchart illustrating a collaborative process operating among a physiological sensor 600, a terminal device 100, a central server 200 and an environmental sensor 700 according to one embodiment of the present disclosure. The terminal device 100 can itself be a specific sensor device in addition to a gateway device for transmitting the data to the central server 200. According to another embodiment of the present disclosure, the terminal device 100 is capable of data processing that can be used to implement an edge-computing device of the health care prediction and warning system, so that the prediction model can be simplified and still serving care and warning prediction based on the received sensing data.

The physiological sensor 600 can represent terminal one or more sensors used to sense physiological status of the care recipient, and the environmental sensor 700 can represent any sensor that is able to sense environmental status, such as a thermometer, a hygrometer, an image sensor, or an air composition and quality sensor as shown in FIG. 2. When the system is in operation, the physiological sensor 600 and the environmental sensor 700 can constantly operate to continuously generate data. In certain embodiments of the present disclosure, the terminal device 100 can continuously receive sensing data that is used to sense the physiological status of the care recipient generated by the physiological sensor 600 (step S601) so as to form the continuous data. The continuous data is firstly stored in a memory of the terminal device 100. The continuous data can be initially processed, including initially determining the physiological status (step S603). The terminal device 100 can periodically or continuously upload the physiological data to the central server 200 according to scheduled time points (step S605).

Further, in one further embodiment of the present disclosure, the terminal device 100 that can perform edge computing for the health care prediction and warning system can load the various prediction indicators that are obtained by a learning process performed in the health care prediction and warning system and/or an individualized care-taking warning prediction model that is obtained by a training process performed in the system. The individualized physiological prediction model can be obtained through the training process with different categories of data with respect to individuals, races, regions and/or genders. Therefore, the terminal device 100 can initially determine the physiological status of the care recipient. If the physiological status of the care recipient meets a specific condition (e.g., requiring further analysis or unable to predict), the data should be uploaded to the central server 200 for further processing.

After the central server 200 receives the continuous data or the labeled non-continuous data from the terminal device 100, the data is under processing and interpretation. For example, the data can be processed by the care-taking warning prediction model for determining the physiological status of the care recipient according to characteristics of the data (step S607). The decision rule can be referred to the flow shown in FIG. 5. The main concept of the decision rule is that a corresponding process will be performed when an analysis result of the physiological data meets one of the criteria. In an exemplary example, when the care-taking warning prediction model predicts the physiological status of the care recipient based on the continuous data, the prediction result is then transmitted to the terminal device 100, and the terminal device 100 displays the result.

Further, under another criterion, when it is determined that additional data is required after the physiological data is processed by the care-taking warning prediction model, the central server 200 generates and transmits an instruction for requesting the additional data to the terminal device 100 (step S609). The terminal device 100 then performs the instruction for providing the additional data (step S611). The additional data to be transmitted can be the physiological data that is generated by a specific physiological sensor 600 within a designated time period (step S613). The additional data can also be the physiological data that is generated within a same time period or another time period before or after a designated time period.

Next, the central server 200 uses the prediction indicators or the care-taking warning prediction model to process the continuous data that is continuously or periodically received from the terminal device 100 and acquires the additional non-continuous data based on the decision rule for effectively determining the physiological status of the care recipient. Furthermore, more additional data can still be required under some specific conditions. In addition to repeating the above steps for acquiring the physiological data, the environmental data can be required (step S615). As the process shows, in the central server 200, an instruction for requesting the environmental data is generated in accordance with the decision rule and transmitted to the environmental sensor 700 or a specific device (step S617), and the environmental sensor 700 transmits the environmental data that is generated within a specific time period to the central server 200 in response to the instruction (step S619). For example, the time period of the environmental data to be transmitted can be the same time period that the physiological data processed by the central server 200 is generated.

After that, the central server 200 is configured to perform care-taking warning prediction based on the physiological data and the corresponding environmental data that are generated within a specific time period through the prediction indicators or by the care-taking warning prediction model (step S621). It should be noted that the above step will be repeated if it is determined that any additional physiological data or environmental data is required. When the physiological status of the care recipient can be effectively determined, a prediction result can be generated and transmitted to the terminal device 100 (step S623).

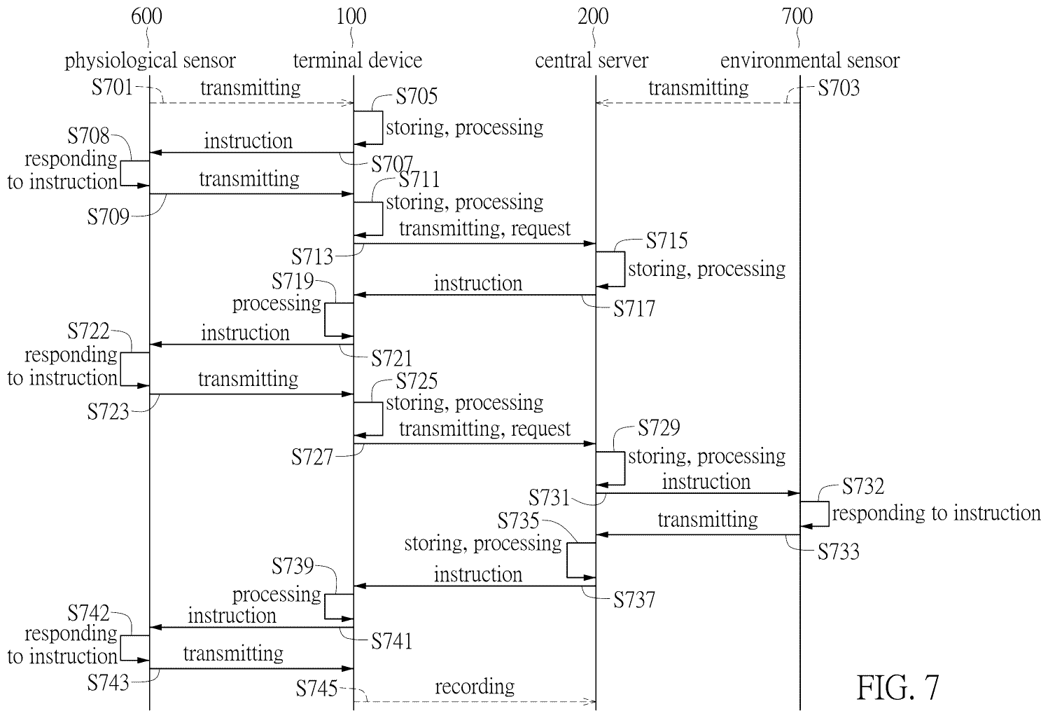

Reference is made to FIG. 7, which is another method for operating the health care prediction and warning system among the sensor 600, the terminal device 100 and the central server 200 according to another embodiment of the present disclosure. The terminal device 100 of the present embodiment implements an edge-computing device that is connected with the physiological sensor 600 that can be one or more of one or more types of physiological sensors. The terminal device 100 can be independently used to embody the health care prediction and warning system. The terminal device 100 can initially and continuously obtain the analyzed physiological data and also can request the additional data from the central server 200 when one of the criteria is met. The additional data requested by the terminal device 100 can be obtained from other sensor such as one or more of one or more types of the environmental sensors 700. Therefore, the terminal device 100 can effectively analyze the features of the physiological data by the care-taking warning prediction model that is operated in the terminal device 100 or through the various prediction indicators that are obtained by a learning process. In certain embodiments of the present disclosure, the care-taking warning prediction model operated in the terminal device 100 or the prediction indicators applied to the terminal device 100 are loaded from the central server 200 and can be individualized for the care recipient so as to initially determine the physiological status of the care recipient.

In the operating method described in FIG. 7, the terminal device 100 can continuously or periodically receive the physiological data from the physiological sensor 600 that is used to sense physiological status of the care recipient (step S701). On the other hand, the central server connected with the terminal device 100 can continuously and periodically receive the physiological data from the terminal device 100 and also continuously and periodically receive the environmental data that is generated by the one or more types of environmental sensors 700 while the environmental sensor 700 continuously sense the environmental status (step S703).

The terminal device 100 stores the received physiological data and analyzes the physiological data by the care-taking warning prediction model or through the various prediction indicators so as to obtain the features of the physiological data and confirm the physiological status of the care recipient (step S705). In the process of processing the physiological data, the features of the physiological data are referred to for determining whether any of the multiple criteria that the physiological sensor 600 or the central server 200 is requested to provide the additional data is met.

Since it is determined that, according to the decision rule, the additional data is required when one of the multiple criteria is met in accordance with the features of the physiological data, an instruction for requesting the additional data is generated and transmitted to the physiological sensor 600 or the central server 200. When the additional data is received from the physiological sensor 600 or the central server 200, the terminal device 100 can effectively determines the physiological status of the care recipient by the care-taking warning prediction model or through the various prediction indicators.

According to the embodiment of the flow shown in FIG. 7, the terminal device 100 relies on an analysis result of the physiological data to determine that the criterion of requiring the physiological sensor 600 to transmit the physiological data that is generated within a specific time period (e.g., the time period before or after the current time) is met, an instruction is generated and transmitted to the physiological sensor 600 (step S707). The physiological sensor 600 then responds to the instruction (step S708) and transmits the physiological data within the designated time period to the terminal device 100 in response to the instruction (step S709). Afterwards, the terminal device 100 cam store the received physiological data that can be provided for the care-taking warning prediction model to analyze or to be analyzed through the prediction indicators (step S711).

It should be noted that, as exemplified in the above embodiment of the decision rule made in the central server 200, the terminal device 100 of the present example has a similar decision rule, which is referred to for deciding a flow to be operated in compliance with one of criteria meeting the physiological status of the care recipient according to features of the physiological data. In the meantime, when one of the criteria is met, the central server 200 is required to analyze the physiological data, a request is issued for transmitting the physiological data within a specific time period to the central server 200 (step S713). Next, the central server 200 stores the data and processes the request transmitted by the terminal device 100 (step S715). After the central server 200 processes the data by the care-taking warning prediction model or through the various prediction indicators, an instruction can be transmitted to the terminal device 100 for requesting the physiological data within a specific time period when a specific criterion is met (step S717). The terminal device 100 then processes the instruction (step S719) and also issues an instruction to the physiological sensor 600 (step S721) in order to request for the physiological data within the time period. After the physiological sensor 600 responds to the instruction (step S722), the physiological data within the time period is transmitted to the terminal device 100 (step S723).

In the meantime, the terminal device 100 stores the additional data. After the additional data is processed by the terminal device 100 (step S725), the terminal device 100 again transmits the physiological data and a request to the central server 200 while the criterion that requests the central server 200 to provide the additional data is met (step S727). After that, the central server 200 stores the data transmitted by the terminal device 100 and processes the data in accordance with the request (step S729). The terminal device 100 can then issue an instruction, either based on the analysis result generated by the care-taking warning prediction model operated in the central server 200 or through the various prediction indicators, to the environmental sensor 700 (step S731). The environmental sensor 700 responds to the instruction (step S732), that is to transmit the environmental data within the specific time period to the central server 200 in response to instruction (step S733). The central server 200 stores the environmental data and then processes the environmental data (step S735) so as to determine the physiological status of the care recipient and then determines if any of the multiple criteria is met. The decision rule is again referred to for deciding following process.

As the present embodiment teaches, the central server 200 transmits an instruction, or accompanied with the environmental data within a specific time period, to the terminal device 100 (step S737), and the terminal device 100 processes the overall received data (step S739). The terminal device can synchronously label the continuous data and the non-continuous data before and after the additional data is transmitted to the terminal device 100, or synchronously label the continuous data and the non-continuous data with the same time period or within a related time period when the additional data is received. Therefore, the physiological status of the care recipient can be effectively predicted. Alternatively, through continuous operations and decisions, repeating the steps of issuing the instruction to the physiological sensor 600 for requesting for the additional data (step S741), the physiological sensor 600 responding to the instruction (step S742), and the additional data being transmitted to the terminal device 100 (step S743) can implement the functionalities of the health care prediction and warning system. Next, as the system demands, the terminal device 100 relies on a specific rule (or periodically) to upload its stored data and processing records to the central server 200 (step S745). The data recorded in the central server 200 can be stored to a database and also can be applied to a subsequent machine learning process.

According to the above embodiments of the health care prediction and warning system and the operating method, a terminal care system capable of predicting physiological status of a care recipient, while cooperated with a central server, can be implemented, some related examples are as follows.

According to knowledges established in clinical medicine, there are three typical signs to myocardial infarction, that are persistent chest pain, chest tightness, and dyspneic respiration. Once the myocardial infarction occurs, the pain often occurs in the front chest and the left side of the chest, or even extends to the left shoulder, upper back, neck and inside of the left arm. Sometimes symptoms of myocardial infarction may also appear in the midst of chin, breast bones and navel. More, myocardial infarction can also appear as weakness, sweating, dizziness, vomiting, unstable heartbeat or falling into coma. The terminal care system implemented by the health care prediction and warning system can predict possible problems before the disease occurs. The physiological sensors of the system can continuously measure chronic disease symptoms such as hypertension, diabetes and hyperlipidemia of the care recipient. When the health care prediction and warning system determines whether any of the criteria is met based on the physiological data of the sensors, the system can request the terminal care system to provide additional sensing data such as electrocardiogram, blood indexes and cardiac biochemical indicators. Therefore, the artificial intelligence model operated in the system can generate a prediction result based on the characteristics of changes of the data and transmit the prediction result to the terminal device for achieving the purpose of advance reminder to prevent myocardial infarction.

One further example is septicemia, which is a very complex disease that often occurs to the patients with weak immune systems, such as the elderly, cancer patients and patients with organ dysfunction or immune deficiency. Symptoms of septicemia are such as body temperature is greater than 38.5 degrees or less than 35 degrees Celsius, heart rate is greater than 90 beats per minute and breathing rate is greater than 20 times per minute, a partial pressure of carbon dioxide in arterial blood is less than 32 mmHg, and a quantity of white blood cells in the blood is greater than 12,000 or less than 4,000 per cubic millimeter. The signals of the above symptoms can be sensed by the sensors at the care recipient's end. An artificial intelligence model operated in the health care prediction and warning system, cooperated with the central server, can make an initial determination according to the data generated by one sensor, such as the quantity of white blood cells in the blood, and then request the terminal device to provide additional data when one of the criteria is met. The additional data for the present example can be body temperatures and heart rate data, or data in other time period. The system can effectively determine if it has possibility of occurring physiological state of septicemia.

In conclusion, the health care prediction and warning system of the present disclosure provides the sensors with multimodal sensing capabilities and can integrate the data generated by various sensors. The data is such as images, sounds, physiological indicators and environmental data. The various sensors should be capable of continuously collecting diverse data without hindrance. The health care prediction and warning system allows the data that is generated by a single-purpose sensor or the sensor that can only sense single type of data to accomplish more complete care functions by the terminal care system through a learning process of an artificial intelligence technology. More, the health care prediction and warning system can depend on low-intrusive and portable sensors and try to use a non-contact design to minimize interferences to the care recipient's daily life with the help of artificial intelligence technology. It should be noted that the miniaturized and lightweight sensors with advantages of long battery life and long-term alertness can be used in the health care prediction and warning system, and it is also advantageous for the system to save data flow loading when only the designated data in compliance the criteria are required in the process. Furthermore, the health care prediction and warning system of the present disclosure is more suitable for the general caregivers or the care recipient to operate since it successfully reduces the threshold for implementing a terminal care system and make the configurations and applications of the sensors easier to be deployed.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for operating a health care prediction and warning system, operated in a central server, comprising:
establishing a connection between the central server and a terminal device;
continuously or periodically receiving physiological data transmitted from the terminal device via the connection;
analyzing the physiological data for acquiring features of the physiological data so as to confirm physiological status of a care recipient and determine whether the features of the physiological data meet any of criteria set in the central server;
wherein, when the central server determines that additional data is required according to the features of the physiological data, an instruction for requesting the additional data is generated and transmitted to the terminal device for enabling the terminal device to transmit the additional data to the central server; and the central server determines the physiological status of the care recipient by a care-taking warning prediction model operated in the central server or through various prediction indicators that are obtained by a learning method.

2. The method according to claim 1, wherein the physiological data that are continuously received by the central server comprises continuous physiological data and non-continuous physiological data that are uploaded in multiple stages or by time sharing.

3. The method according to claim 2, wherein the terminal device embodies one of edge-computing devices of the health care prediction and warning system and initially determines physiological status of the care recipient by loading the various prediction indicators learned by the central server or by the care-taking warning prediction model that is individualized.

4. The method according to claim 2, wherein, in the central server, the care-taking warning prediction model is operated to obtain features from the continuously-received physiological data at every time point or in each time interval, and calculate a probability of each of health risks, so as to determine physiological status of the care recipient.

5. The method according to claim 4, wherein the central server relies on a decision rule to decide a flow to be operated in compliance with one of multiple criteria meeting the physiological status of the care recipient according to features of the physiological data; wherein the multiple criteria at least comprise:
a first criterion, for generating a result of predicting physiological status of the care recipient through multiple prediction indicators or by the care-taking warning prediction model that analyzes and compares the physiological data based on the physiological data and outputting the result;
a second criterion, for the central server to request the terminal device to transmit the physiological data generated at another time period based on the result that is generated through the multiple prediction indicators or by the care-taking warning prediction model, so that the physiological status of the care recipient is predicted; and
a third criterion, for the central server to request the terminal device to provide a sensing data obtained by another sensor based on the result that is generated through the multiple prediction indicators or by the care-taking warning prediction model so as to predict the physiological status of the care recipient.

6. The method according to claim 1, wherein the central server requests the terminal device to transmit the physiological data that includes the physiological data that is generated by a specified sensor at a same time period, or the physiological data that is generated by the same sensor or the specified sensor at another time period before or after the same time period.

7. The method according to claim 1, wherein the terminal device is a sensor device or the terminal device connects with one or more sensor devices, which is used to obtain the physiological data and/or environmental data, and upload the physiological data and/or the environmental data to the central server; and, in the central server, the physiological status of the care recipient is determined by the care-taking warning prediction model or through the multiple prediction indicators based on the physiological data and the environmental data, so as to generate a prediction result and transmit the prediction result to the terminal device.

8. The method according to claim 1, wherein, for the additional data that the terminal device transmits to the central server, the central server synchronously labels continuous data and non-continuous data that are obtained before and after requesting for the additional data, or synchronously labels the continuous data and the non-continuous data that are generated at a same time period or a related time period for forming individualized data for the care recipient; wherein an artificial intelligence technology is used to learn features from the continuous data and the non-continuous data so as to establish the care-taking warning prediction model that is individualized or the multiple prediction indicators.

9. A method for operating a health care prediction and warning system, implemented by a terminal device, comprising:
continuously or periodically receiving physiological data transmitted by one or more physiological sensors;
analyzing the physiological data for acquiring features of the physiological data so as to confirm physiological status of a care recipient by a care-taking warning prediction model operated in the terminal device or through various prediction indicators that are obtained by a learning method; and
determining whether meeting one of the multiple criteria for requesting the one or more physiological sensors to provide additional data according to features of the physiological data, or requesting a central server to provide the additional data;
wherein, when the terminal device determines that additional data is required as meeting one of the multiple criteria according to the features of the physiological data, an instruction for requesting for the additional data is generated for driving the one or more physiological sensor or the central server to provide the additional data to the terminal device, so that the terminal device is able to determine the physiological status of the care recipient by the care-taking warning prediction model operated in the terminal device or through various prediction indicators that are obtained by a learning method.

10. The method according to claim 9, wherein the terminal device embodies one of edge-computing devices of the health care prediction and warning system and initially determines physiological status of the care recipient by loading the various prediction indicators learned by the central server or by the care-taking warning prediction model that is individualized.

11. The method according to claim 10, wherein the terminal device relies on a decision rule to decide a flow to be operated in compliance with one of criteria meeting the physiological status of the care recipient according to features of the physiological data, and the criteria at least comprise:
a first criterion, for generating a result of predicting physiological status of the care recipient through multiple prediction indicators or by the care-taking warning prediction model that analyzes and compares the physiological databased on the physiological data and outputting the result;
a second criterion, for the central server to request the terminal device to transmit the physiological data generated at another time period based on the result that is generated through the multiple prediction indicators or by the care-taking warning prediction model, so that the physiological status of the care recipient is predicted; and
a third criterion, for the central server to request the terminal device to provide a sensing data obtained by another sensor based on the result that is generated through the multiple prediction indicators or by the care-taking warning prediction model so as to predict the physiological status of the care recipient.

12. The method according to claim 11, wherein, in the terminal device, the features of the physiological data are referred to for determining whether the features meet the criterion to request the central server to provide the additional data, and the central server is requested to transmit environmental data obtained from one or more environmental sensors at a specific time period when the criterion is met.

13. The method according to claim 9, wherein, for the additional data transmitted to the terminal device, the terminal device synchronously labels continuous data and non-continuous data that are obtained before and after requesting for the additional data, or synchronously labels the continuous data and the non-continuous data that are generated at a same time period or a related time period.

14. A health care prediction and warning system, comprising:
a central server that operates a care-taking warning prediction model and continuously or periodically receives physiological data transmitted from a terminal device via a connection between the central server and the terminal device;
wherein, after the physiological data is analyzed to obtain features of the physiological data, the central server confirms physiological status of a care recipient and determines whether the features of the physiological data meets any of criteria set in the central server; wherein, when the central server determines that additional data is required according to the features of the physiological data, an instruction for requesting the additional data is generated and transmitted to the terminal device so as to enable the terminal device to transmit the additional data to the central server, and the central server determines the physiological status of the care recipient by a care-taking warning prediction model operated in the central server or through various prediction indicators that are obtained by a learning method.

15. The health care prediction and warning system according to claim 14, wherein the terminal device embodies one of edge-computing devices of the health care prediction and warning system, and initially determines physiological status of the care recipient or provides follow-up care suggestions by loading the multiple prediction indicators learned by the central server or by the care-taking warning prediction model that is individualized.

16. The health care prediction and warning system according to claim 14, wherein the terminal device beside the care recipient is a sensor device or connects with one or more sensors and the terminal device receives and stores data that is generated by the sensor device or the one or more sensors and comprises the physiological data that are uploaded in multiple stages or by time sharing.

17. The health care prediction and warning system according to claim 16, wherein the sensor device or any of the one or more sensors is a sensor with a single sensing capability or a sensor with multimodal sensing capabilities; and the physiological data are processed with encoding, encryption and format conversion by a processing circuit of the terminal device so as to generate continuous data or non-continuous data to be transmitted to the health care prediction and warning system.

18. The health care prediction and warning system according to claim 16, wherein, in the central server, the care-taking warning prediction model is operated to obtain features from the continuously-received physiological data at every time point or in each time interval, and calculate a probability of each of health risks, so as to determine physiological status of the care recipient.

19. The health care prediction and warning system according to claim 18, wherein the central server relies on a decision rule to decide a flow to be operated in compliance with one of multiple criteria meeting the physiological status of the care recipient according to features of the physiological data, wherein the multiple criteria at least comprise:
a first criterion, for generating a result of predicting physiological status of the care recipient through multiple prediction indicators or by the care-taking warning prediction model that analyzes and compares the physiological databased on the physiological data and outputting the result;
a second criterion, for the central server to request the terminal device to transmit the physiological data generated at another time period based on the result that is generated through the multiple prediction indicators or by the care-taking warning prediction model, so that the physiological status of the care recipient is predicted; and
a third criterion, for the central server, based on the result that is generated through the multiple prediction indicators or by the care-taking warning prediction model, to request the terminal device to transmit the physiological status that is generated by a specified sensor at the same time period, or the physiological data that is generated by the same sensor or the specified sensor at another time period before or after the same time period so as to predict the physiological status of the care recipient.

20. The health care prediction and warning system according to claim 19, wherein, for the additional data transmitted to the central server from the terminal device, the central server synchronously labels continuous data and non-continuous data that are obtained before and after time to requesting for the additional data, or synchronously labels the continuous data and the non-continuous data that are generated at a same time period or a related time period.
